(19) United States
(12) Patent Application Publication
(10) Pub. No.: US 2015/0018648 A1
(43) Pub. Date: Jan. 15, 2015

(54) SYSTEMS AND METHODS FOR MONITORING PATIENTS ON VENTILATION

(71) Applicant: Covidien LP, Mansfield, MA (US)

(72) Inventors: Robert T. Boyer, Longmont, CO (US); Gary S. Milne, Louisville, CO (US)

(21) Appl. No.: 13/941,817

(22) Filed: Jul. 15, 2013

(51) Int. Cl. A61M 16/10; A61B 5/00; A61M 16/00; A61B 5/1455

(52) U.S. Cl. CPC A61M 16/1005; A61B 5/14551; A61B 5/7282; A61B 5/742; A61B 5/746; A61B 5/7246; A61M 16/0051; A61M 16/0057; A61M 16/0003; A61B 5/4839; A61M 2016/1025
USPC 600/323; 128/204.23

(57) ABSTRACT

A system for monitoring a patient on ventilation including a ventilator system, a pulse oximeter, an information processing system, and a display device. The ventilator system is configured to provide mechanical ventilation in accordance with an FiO₂ setting. The pulse oximeter is configured to measure SpO₂. The information processing system is communicatively coupled to the ventilator system and the pulse oximeter and is configured to receive, process, and output FiO₂ setting information and SpO₂ data from the ventilator system and the pulse oximeter, respectively. The display device is configured to display the FiO₂ setting information and SpO₂ data graphically as a function of time.

### TECHNICAL FIELD

The present disclosure relates to patient monitoring and, more particularly, to systems and methods for monitoring patients on mechanical ventilation.

### BACKGROUND

It has been estimated that more than 300,000 patients receive mechanical ventilation in the United States each year. Mechanical ventilation is often an essential, life-saving therapy for critically ill patients and patients experiencing respiratory failure. However, despite the obvious benefits, patients on mechanical ventilation are at an increased risk for complications such as Ventilator-Associated Pneumonia (VAP), sepsis, Acute Respiratory Distress Syndrome (ARDS), pulmonary embolism, barotraumas, and pulmonary edema. These complications may necessitate extended ventilation use, longer hospital stays, increased costs, and/or may increase the risk of disability or death.

Recently, the Centers for Disease Control and Prevention (CDC) has established a protocol for the surveillance and reporting of so-called Ventilator Associated Events (VAEs). This Ventilator Associated Event Protocol is designed to facilitate the gathering of data related to known occurrences of VAEs so that the effectiveness of prevention strategies can be assessed.

### SUMMARY

The present disclosure relates to systems and methods for monitoring a patient on ventilation. Systems provided in accordance with the present disclosure include, for example, a ventilator system, a pulse oximeter, an information processing system, and a display device. The ventilator system is configured to provide mechanical ventilation in accordance with an FiO₂ setting. The pulse oximeter is configured to measure SpO₂. The information processing system is communicatively coupled to the ventilator system and the pulse oximeter and is configured to receive, process, and output FiO₂ setting information and SpO₂ data from the ventilator system and the pulse oximeter, respectively. The display device is configured to display the FiO₂ setting information and SpO₂ data graphically as a function of time.

The present disclosure is advantageous in that it provides for greater visibility into the correlation and relationship between the FiO₂ setting of the ventilator and patient SpO₂ data. Such a feature provides early awareness and real-time notifications indicating a potential need to adjust the FiO₂ setting; relevant past, present, and trending SpO₂ data; and/or that the patient may be trending towards a particular condition, e.g., a potential VAE. The present disclosure is further advantageous in the ability to provide a feedback-based control loop that adjusts the FiO₂ setting based upon the patient SpO₂ data in accordance with customizable threshold settings. The feedback-based control loop may include threshold settings different and independent from threshold settings utilized in the notification system.

Certain embodiments of the present disclosure may include some, all, or none of the above advantages and/or one or more other advantages readily apparent to those skilled in the art from the drawings, descriptions, and claims included herein. Moreover, while specific advantages have been enumerated above, the various embodiments of the present disclosure may include all, some, or none of the enumerated advantages and/or other advantages not specifically enumerated above.

### BRIEF DESCRIPTION OF THE DRAWINGS

The present disclosure and its various aspects and features are described hereinbelow with reference to the accompanying drawings, wherein:

FIG. 1 is a schematic illustration of a patient care and monitoring system provided in accordance with the present disclosure;

FIG. 2 is a schematic illustration of a hardware and software configuration for use with the system of FIG. 1;

FIG. 3 illustrates a display screen provided in accordance with the present disclosure, as presented to a user;

FIG. 4 illustrates a setting screen provided in accordance with the present disclosure, as presented to a user; and

FIG. 5 illustrates another display screen provided in accordance with the present disclosure, as presented to a user.

### DETAILED DESCRIPTION

Provided in accordance with the present disclosure are systems and methods for monitoring a patient on a mechanical ventilator. Specifically, the present disclosure provides for monitoring, compiling, and outputting, e.g., sending feedback-based control signals, visually displaying, providing notifications (audible, text, etc.), generating reports, etc., past, current, and/or trending information indicating the relationship between the “Fraction of inspired Oxygen” (FiO₂) setting of the ventilator and the patient's measured percent oxygen saturation of hemoglobin in arterial blood (SpO₂). As will be detailed below, adjustment of the FiO₂ setting of the ventilator is often determined in accordance with the patient's measured SpO₂, e.g., if SpO₂ is too low, the FiO₂ level may be increased while, if SpO₂ is above a particular threshold, the FiO₂ level may be decreased. Thus, by monitoring SpO₂ levels; visually displaying past, current, and trending SpO₂ information; utilizing such SpO₂ information in a feedback loop (open or closed); and/or providing alerts based on SpO₂ information, the current condition and/or trending condition of a patient on mechanical ventilation can be readily ascertained and, in response, proper steps may be taken automatically via a feedback-based system or manually facilitated by an alert-based system. In addition, by monitoring SpO₂ information after a change in the FiO₂ level, a patient's response, condition status, and/or trend can also be readily determined.

In one particular implementation, the aspects and features of the present disclosure may be incorporated into monitoring systems and methods for monitoring a patient on mechanical ventilation for risks of VAEs and for observing the occurrence of VAEs. Certain parameters, conditions, and/or sensed data have been found to indicate an increased risk that a patient may suffer a VAE, that a VAE is ongoing, and/or that a VAE has occurred. As will be detailed below, such parameters, conditions, and/or data include, but are not limited to, the ventilator settings FiO₂ and Positive-End Expiratory Pressure (PEEP), body temperature, white blood cell (WBC) count, drug administration schedule information, and/or the duration of time the patient has been on the ventilator. Past information, current information, and/or trending information regarding these parameters, conditions, and/or sensed data can be utilized to determine a patient's risk of suffering a VAE as well as for gathering data related to an occurring or previously-occurred VAE. Monitoring, compiling, and outputting, e.g., via visually displaying, providing notifications (audible, text, etc.), generating reports, etc., past, current, and trending information can be utilized to facilitate a user in readily and early ascertaining a patient's current status and risk of suffering a VAE, whether the patient is currently experiencing a VAE, and/or whether the patient has experienced a VAE. A more detailed description of systems and methods suitable for this purpose are described in U.S. patent application Ser. No. 13/768,769 entitled “SYSTEMS AND METHODS FOR MONITORING PATIENTS FOR RISK OF VENTILATOR ASSOCIATED EVENTS,” filed on Feb. 15, 2013, the entire contents of which are hereby incorporated by reference herein. The above-noted aspects and features of the present disclosure, which will be described in greater detail below, may be utilized concurrently; that is, the present disclosure provides for the ability to both utilize an SpO₂-based feedback and/or alert system for monitoring a patient on mechanical ventilation in general and also utilize the SpO₂ information in the specific application of monitoring, alerting, and reporting VAEs, while maintaining separate control settings for each.

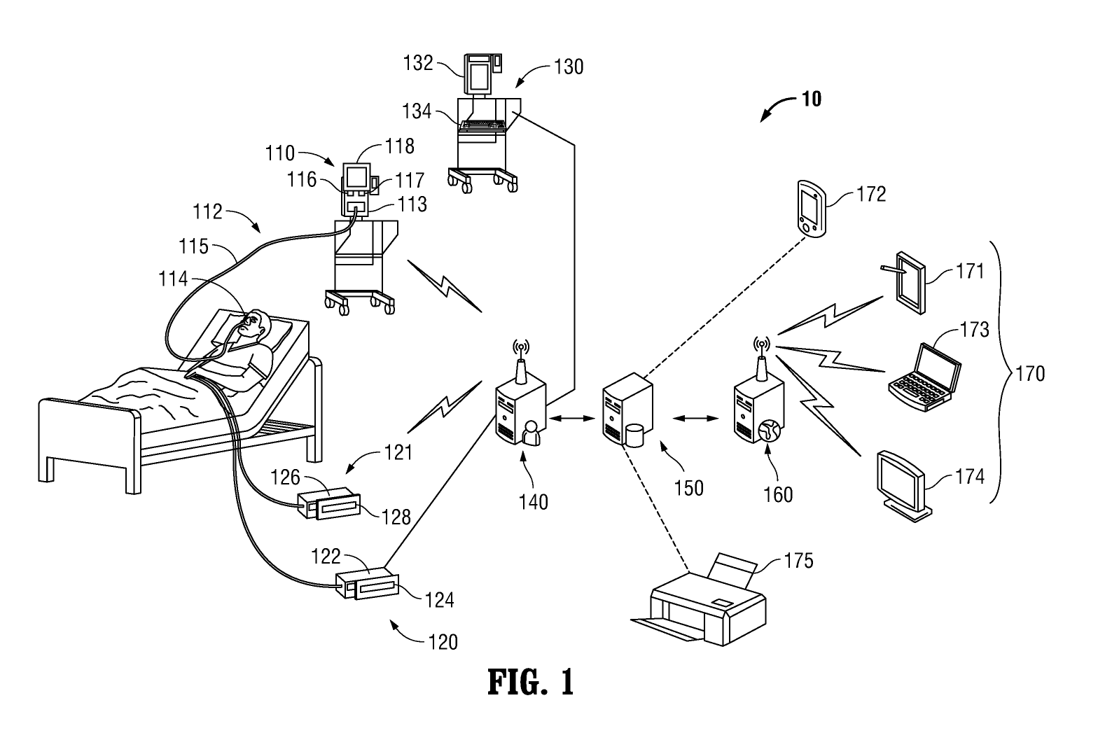

Referring to FIG. 1, an exemplary system provided in accordance with the present disclosure is shown generally identified by reference numeral 10. System 10 includes one or more patient care devices 110, one or more patient monitoring devices 120, 121, one or more bedside devices 130, one or more data servers 140, one or more application servers 150, one or more web servers 160, and one or more remote devices 170. For the purposes herein, exemplary system 10 is generally described, although the aspects and features of the present disclosure may be implemented, incorporated, or utilized with any other devices, systems, and combinations thereof.

The one or more patient care devices 110 may include, for example, a ventilator system 112. Ventilator system 112 may be any suitable ventilator system, e.g., the Puritan Bennett™ 840 Ventilator System sold by Covidien LP of Boulder, Colo., USA, and generally includes a bedside ventilator unit (“ventilator”) 113, a patient interface member 114, e.g., an artificial airway (for invasive ventilation) or mask (for noninvasive ventilation), and a ventilator circuit 115, e.g., an inspiratory and expiratory limb, interconnecting the ventilator 113 and patient interface member 114. The ventilator 113 controls the supply of oxygenated air to the patient and includes at least a first input 116 for allowing a user to set a desired FiO₂ level and a second input 117 for allowing a user to set a desired PEEP. Ventilator 113 may further include a display 118 for displaying relevant data relating to ventilator system 112 and/or the patient. Ventilator 113 may be coupled to one or more servers 140, 150, 160, e.g., data server 140, either wirelessly or via a wired connection. Ventilator 113 may include any suitable software, firmware, and hardware for the above purposes.

The one or more patient monitoring devices 120, 121 may include any suitable device(s) for visual monitoring, audible monitoring, monitoring of physical characteristics, physiological conditions, other measurable characteristics or conditions, etc. For example, patient monitoring devices 120, 121 may be a temperature sensor 122 and a pulse oximeter 126, respectively. Temperature sensor 122 may be configured to display temperature data on a visual display 124 thereof and/or to relay temperature data to one or more servers 140, 150, 160, e.g., data server 140. Temperature sensor 122 may be wirelessly coupled to data server 140, or may be coupled to data server 140 via a wired connection.

Pulse oximeter 126 may be configured to measure the SpO₂ at a particular location (or locations) on the patient and to relay the same to portable monitoring device 121. Pulse oximeter 126 may be configured for obtaining an SpO₂ reading at specific intervals. The intervals at which readings are taken may be constant, e.g., one reading every second, or may be taken manually upon request. Portable monitoring device 121 may be configured to process and display the SpO₂ data on a visual display 128 thereof and/or may be configured to relay the SpO₂ data to one or more servers 140, 150, 160, e.g., data server 140. This may be done wirelessly as shown with respect to portable monitoring device 121, or as a wired connection as shown with respect to portable monitoring device 120. Additional monitoring devices may similarly be employed to monitor other characteristics, conditions, or to otherwise monitor the patient and to process the patient data, display the patient data, and/or relay the patient data to data server 140. The patient monitoring devices may be wirelessly coupled to data server 140, or may be coupled to data server 140 via a wired connection. The patient monitoring devices may include any suitable software, firmware, and/or hardware for the above-noted purposes.

Bedside device 130 may include a display 132 and a user input 134, e.g., a touch-screen display or keyboard and mouse. Bedside device 130 is employed to display relevant parameters, conditions, sensed data, and/or other patient information on display 132 at a patient's bedside. Bedside device 130 is further configured to receive user input data via user input 134 for display on display 132 and/or for transmission to data server 140 (and/or the other servers 150, 160). Exemplary information input into bedside device 130 may include medical care information (e.g., the patient's drug administration schedule), control parameters, other measured data, and/or biographical or other observed data/notes. Bedside device 130 may be wirelessly coupled to data server 140, or may be coupled to data server 140 via a wired connection. Bedside device 130 may include any suitable software, firmware, and hardware for the above purposes. Alternatively or additionally to bedside devices 130, patient monitoring devices 120, 121, and patient care devices 110, information relating to the patient may be automatically pulled from another system, e.g., a patient's Electronic Medical Record (EMR), an Admission, Discharge, and Transfer (ADT) electronic file, lab data, etc. This information may be utilized directly and/or may be used in determining any of the other information, e.g., control parameters or other settings, as will be detailed below.

Data server 140, application server 150, and web server 160 are coupled to one another and between remote devices 170 and the local devices, e.g., ventilator system 112, temperature sensor 122, pulse oximeter 126, and bedside device 130, for storing, processing, and/or transmitting information therebetween. More specifically, one or more of servers 140, 150, 160, e.g., data server 140, are configured to store information, e.g., the parameters, conditions, sensed data, reports, and/or other information, in a database and to process the information. Servers 140, 150, 160 are further configured to cooperate with one another to transmit information between the servers 140, 150, 160, other systems, e.g., a patient's EMR, an ADT electronic file, lab data, etc., ventilator system 112, temperature sensor 122, pulse oximeter 126, bedside device 130, and/or remote devices 170. Servers 140, 150, 160 may include any suitable software, firmware, and hardware for these purposes and may establish the above-described communication via wired and/or wireless communication.

Remote devices 170 request and receive information, e.g., the parameters, conditions, sensed data, and/or other information, process the information, if needed, and display the information to a user, e.g., via a display monitor, user interface, browser, and/or application running on the remote device 170, or otherwise output the information to a user, e.g., print a generated report containing the information. Remote devices 170 may further be configured to receive input from a user, e.g., to input information, control the display or output of the information, set parameters, reset notifications, etc. Information input into remote devices 170 may include medical care information (e.g., the patient's drug administration schedule), control parameters, other measured data (e.g., the patient's WBC), and/or biographical or observed data. Remote devices 170 may include one or more tablet PCs 171, smartphones 172, laptop computers 173, display monitors 174, printers 175, or other suitable devices and may incorporate any suitable software, firmware, and hardware for the above purposes.

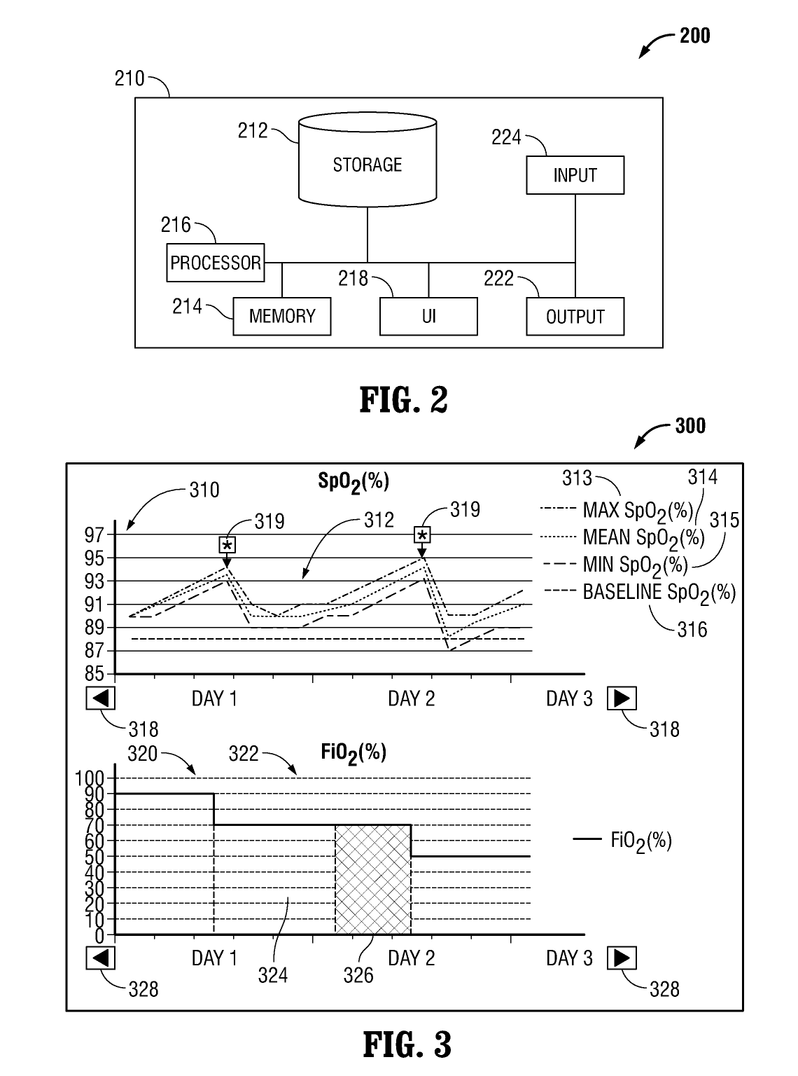

Turning now to FIG. 2, in conjunction with FIG. 1, one configuration of hardware and software components for receiving/transmitting information, e.g., control parameters, conditions, sensed data, and/or other information, processing the information, receiving user input, and/or displaying the information or otherwise outputting the information in accordance with the present disclosure is shown generally identified by reference numeral 200. Configuration 200 may be embodied within one or more of patient care devices 110, patient monitoring devices 120, 121, bedside devices 130, servers 140, 150, 160, and remote devices 170, or may be implemented across one or more of patient care devices 110, patient monitoring devices 120, 121, bedside devices 130, servers 140, 150, 160, and remote devices 170. That is, receiving/transmitting the information and user input, processing the information, and outputting the information for control, display, or other output may be performed locally, e.g., at one of patient care devices 110, patient monitoring devices 120, 121, bedside devices 130; on one or more servers 140, 150, 160 for distribution to the various devices 110, 120, 121, 130, 170, e.g., across a network; at the remote devices 170 themselves; or in any combination of the above. For the purposes of simplicity, configuration 200 will be described herein as embodied in a system 210, keeping in mind that system 210 may be incorporated into or across any or all of the components of system 10.

System 210 generally includes a storage 212, a memory 214, a processor 216, a user interface (UI) 218, an output 222, and an input 224. Storage device 212 may include any suitable component(s) operable for storing information received via input 224, such as, for example, a magnetic disk, flash memory, optical disk, or other suitable data storage device. Memory 214 may include any computer memory, e.g., RAM or ROM, mass storage media, removable storage media, combinations thereof, or any other suitable computer-readable storage medium, storing instructions for causing processor 216 to execute particular functions, e.g., to process the information. Processor 216 may include any suitable component(s), e.g., a central processing unit (CPU), operable to execute instructions stored in memory 214 to process and manipulate information, e.g., stored in storage device 212 or received via input 224, for output to UI 218 or output 222. Processor 216 is further configured to receive, via input 224 and/or UI 218, information, data, and/or control parameters for processing and manipulating the information in accordance with user-selected settings and user input. UI 218 functions to output the processed information for visual display, e.g., in graphical and/or numerical form, to the user and/or allows for the input of information, data, setting of parameters, etc., by the user. Output and input 222, 224, respectively, are provided to facilitate communication between system 210 and the other components of system 10. In particular, input 224 is configured to receive information to be processed, e.g., data from ventilator system 112, temperature sensor 122, pulse oximeter 126, and bedside device 130 (or other device where user-input data is provided).

With reference to FIG. 3, in conjunction with FIGS. 1 and 2, as mentioned above, a display screen 300 in accordance with embodiments of the present disclosure is shown displaying exemplary information as output by UI 218. Main display screen 300 may represent display 118 of ventilator system 112 (or other patient care device 110), display 132 of bedside device 130, or a monitor, display, etc. of any of remote devices 170. That is, depending on the configuration of system 10, the user may view display screen 300 on one of the patient care devices 110, patient monitoring devices 120, 121, bedside devices 130, remote devices 170, or any other suitable device. Main display screen 300, as will be detailed below, incorporates the above-described features of monitoring, compiling, and outputting information to graphically represent the FiO₂ setting of the ventilator and the patient's SpO₂ and to indicate the relationship therebetween.

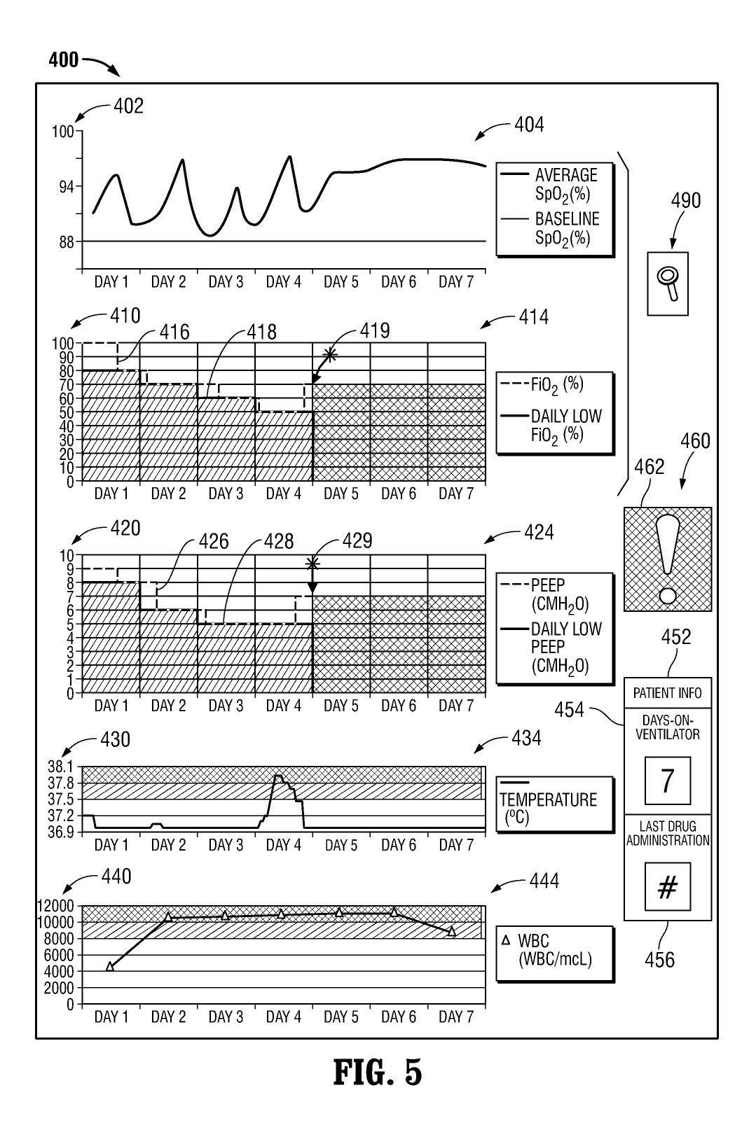

Display screen 300 includes first and second graph image areas 310, 320 for graphically displaying current and trending information relating to SpO₂ and FiO₂, respectively. The orientation, ordering, and/or relative positioning of graph image areas 310, 320 may be selected and/or changed in accordance with user preference. As shown in FIG. 3, the SpO₂ graph 312 and the FiO₂ graph 322 are grouped together with the SpO₂ graph 312 positioned directly above the FiO₂ graph 322. This configuration provides an intuitive layout as SpO₂ information is typically a factor used in determine whether the FiO₂ level should be adjusted and/or may be used to determine the reasoning behind a prior adjustment of the FiO₂ level. Although not shown, patient information and/or other measured or received data may also be displayed on display screen 300, such as the information and data shown on display screen 400 (FIG. 5).

As noted above, first graph image area 310 is configured for displaying an SpO₂ graph 312 representing the patient's measured SpO₂ (as a percentage) as a function of time. SpO₂ measurements may be taken at pre-determined intervals or continuously, e.g., using pulse oximeter 126. SpO₂ measurements received by pulse oximeter 126 are transmitted to processor 216 in real-time (or are transmitted to storage 212, for processing at a later time) for processing and output as a graphical representation for display in first graph image area 310 of display screen 300. More specifically, various discrete data points at pre-determined time intervals, e.g., hourly, are provided via processor 216 for display on first graph image area 310 of display screen 300. For example, as shown in FIG. 3, SpO₂ graph 312 includes a MAX SpO₂ curve 313, a MEAN SpO₂ curve 314, a MIN SpO₂ curve 315, and a baseline SpO₂ line 316. The MAX SpO₂ curve 313 represents the maximum SpO₂ reading within each time interval. The MEAN SpO₂ curve 314 represents the mean or average SpO₂ reading across each time interval. The MIN SpO₂ curve 315 represents the minimum SpO₂ reading within each time interval. The baseline SpO₂ line 316 represents the baseline SpO₂ setting from which low and high limits, alerts, notifications, and/or feedback-based controls are determined. As an alternative to providing a plurality of curves on graph 312, the MEAN SpO₂ curve 314 may be provided with error bars representing the corresponding minimum and maximum SpO₂ values within each time interval. Scroll icons 318 are provided on either side of graph 312 to permit a user to scroll forwards and backwards through the displayed graph 312.

Second graph image area 320 is configured for displaying a FiO₂ graph 322 representing the FiO₂ level (as a percentage) set in ventilator system 112 as a function of time. FiO₂ level information set in ventilator system 112 is transmitted to processor 216 in real-time (or is transmitted to storage 212, for processing at a later time) for processing and output as a graphical representation for display in second graph image area 320 of display screen 300. As FiO₂ is a ventilator setting, rather than a measured variable, the FiO₂ graph 322 is shown including a stepped graph line, rather than a “curve.” As can be appreciated, the steps illustrated by the graph line of graph 322 indicate the points in time where the FiO₂ setting was adjusted. As detailed below and shown in FIG. 5, FiO₂ graph 322 may include both an actual FiO₂ indicator line 416 (the broken line) and a daily low FiO₂ indicator line 418 (the solid line) superimposed on one another. Additionally or alternatively, the FiO₂ graph 322 may include shading or coloring in accordance with a coding protocol. Similarly as above, scroll icons 328 are provided on either side of graph 322 to permit a user to scroll forwards and backwards through the displayed graph 322.

In accordance with the above, as can be appreciated, the patient's SpO₂ readings as compared to the FiO₂ level (and whether there has been a recent change) can be conveniently monitored, in real-time or as new data for these indications is received (whether automatically sensed or manually input), by a user in accordance with the present disclosure by viewing display screen 300. That is, display screen 300 provides current, real-time, or most recent information as well as trend information regarding both a patient's SpO₂ readings and the FiO₂ level. The user is thus able to readily ascertain whether, and in what manner, FiO₂ level changes have impacted patient's SpO₂ and/or, in the converse, whether, and in what manner, FiO₂ level adjustment is recommended based upon the past, current, or trending SpO₂ information. For example, as shown in FIG. 3, upon a decrease in the FiO₂ level set in ventilator system 112, a patient's SpO₂ may initially decreases, stabilizes, and then increase. Such a normal occurrence of a recovering/improving patient can be readily ascertained by viewing display screen 300. Without providing the FiO₂ graph 322 which indicates that the FiO₂ level has been decreased, no reason for the change in SpO₂ would be readily ascertainable. On the other hand, without displaying the SpO₂ graph 312, the user would be unable to readily ascertain how the patient was responding to the decrease in the FiO₂ level.

The above-described display screen 300 not only presents relevant information regarding the levels or values of SpO₂ and FiO₂ relative to each other and in temporal relation to facilitate determinations by a user, but may also provide alerts, color-coding, and status indicators to facilitate such determinations, as detailed below.

Referring still to FIG. 3, with respect to color-coding on display screen 300, where SpO₂ readings are within pre-defined threshold limits, e.g., the low and high threshold limits detailed below, the area 324 under the corresponding portion of the curve of FiO₂ graph 322 may be un-shaded or shaded with a first color or pattern, e.g., green. Upon significant (as determined by pre-defined parameters such as the low and high threshold limits detailed below) trending or changing of the SpO₂ level, but before the FiO₂ level has been adjusted, the area 326 under the corresponding portion of the curve of graph 322 may be shaded with a different, second color or pattern, e.g., red, thus alerting a user that the patient's SpO₂ level is trending towards a condition wherein a potential FiO₂ level adjustment may be needed. It is also contemplated that a plurality of levels of color coding be used to provide more granularity with respect to this alert condition. Color-coded or symbolized indicators 319 on the SpO₂ graph 312 may also be provided to indicate the point in time where the FiO₂ level was adjusted, thus allowing the user to readily ascertain the temporal relation between changes in SpO₂ levels and FiO₂ level adjustments, how the patient is responding to the FiO₂ level adjustment, and/or whether SpO₂ changes are the result of the FiO₂ level adjustment or potentially the result of something unrelated to the interplay between SpO₂ and FiO₂. Color-coding may also be utilized to indicate the SpO₂ level relative to the baseline, on either or both graphs 312, 322.

As mentioned above, a patient's SpO₂ is often used in determining whether to increase, decrease, or maintain the FiO₂ setting of the ventilator. Accordingly, SpO₂ data may be utilized as part of a feedback-based system for automatically adjusting the FiO₂ setting of the ventilator in accordance with parameters defined by the user and/or may be utilized to provide an alert indicating occurrence of a pre-defend condition, that pre-defined parameters have been met, etc., to indicate to the user that adjustment of the FiO₂ setting of the ventilator may be warranted. Additionally or alternatively, the SpO₂ data may be utilized to inhibit adjustment of the FiO₂ setting of the ventilator, e.g., without proper override, or to provide an alert or warning where the FiO₂ setting has been adjusted (or is requested to be adjusted) and adjustment is not determined to be warranted based upon the pre-defined parameters or pre-defined conditions associated with the patient's SpO₂. With respect to these above-noted features, processor 216 may be configured to receive the SpO₂ data from pulse oximeter 126 and the FiO₂ data from ventilator system 112 via input 224, process such data, and provide control signals accordingly, e.g., for adjustment of the FiO₂ setting of the ventilator system 112, to lock ventilator system 112 from adjustment, and/or to display an alert/warning on one or more of the visual displays of system 10. The parameter settings for such automatic adjustments, alerts, and the like are detailed below.

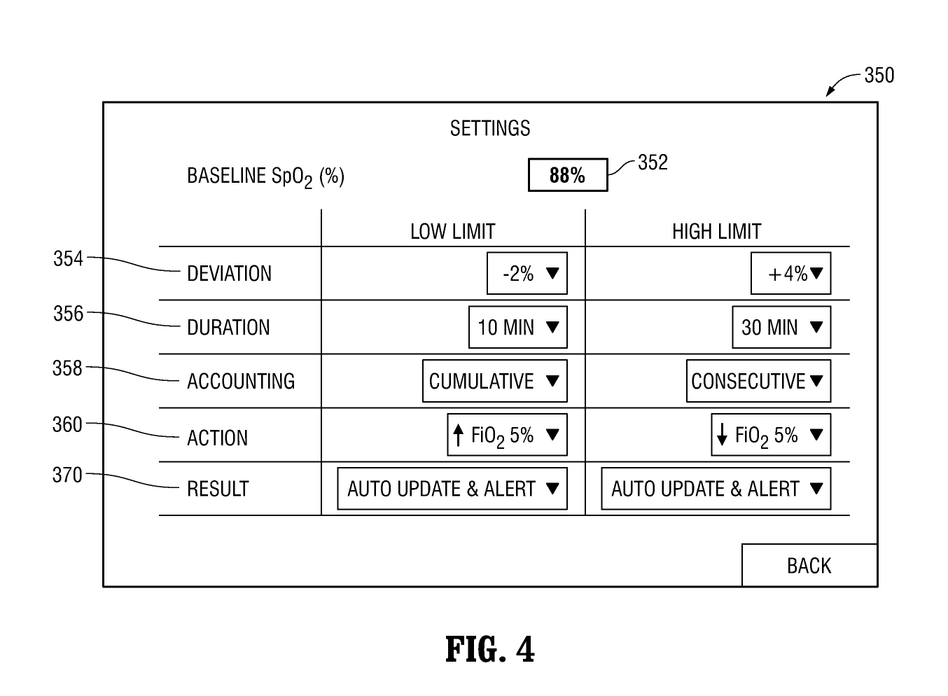

FIG. 4 illustrates an exemplary setting screen 350 as presented to a user for allowing a user to set parameters and/or verify current parameter settings. However, it is envisioned that any suitable setting screen or screens be provided to facilitate such adjustment and/or verification. With additional reference to FIG. 1, setting screen 350 may represent display 118 of ventilator system 112 (or other patient care device 110), display 132 of bedside device 130, or a monitor, display, etc. of any of remote devices 170. That is, depending on the configuration of system 10, the user may view setting screen 350 on one of the patient care devices 110, patient monitoring devices 120, 121, bedside devices 130, remote devices 170, or any other suitable device.

Setting screen 350 includes a baseline SpO₂ setting 352 and low and high limit settings for deviation 354, duration 356, accounting 358, action 360, and result 370. Baseline SpO₂ setting 352 is the metric from which the alerts/notifications and/or adjustments with respect to FiO₂ are made and may correspond to a target SpO₂ level. Baseline SpO₂ setting 352 may be input by a user, may be automatically generated based upon information received via system 10 and processed via processor 216, or may assume a default or one of a plurality of pre-set setting configurations stored in memory 214. The deviation settings 254 indicate the low and high limit SpO₂ thresholds with respect to deviation from the baseline SpO₂ setting 352. For example, as shown in FIG. 4, the low limit deviation threshold is −2%, e.g., an SpO₂ of 86% using a baseline SpO₂ of 88%. The deviation setting 354 is represented using a percentage as the units in that SpO₂ is provided in terms of a percentage, but is an absolute setting, e.g., a 10% negative deviation from an 88% baseline corresponds to a SpO₂ of 78%. Alternatively, the deviation may be provided in relative terms as a percentage of the current SpO₂ value, e.g., where a 10% negative deviation from an 88% baseline corresponds to a SpO₂ of 79.2%. As shown, the deviation thresholds 354 (as well as the other settings) for the low and high limits may be different and may be independently set.

The duration settings 356 correspond to the amount of time the deviation threshold is required to be met, while the accounting setting 358 provides the method for which this amount of time is calculated. For example, as shown in FIG. 4, with respect to the low limit and with the low limit deviation threshold set at −2%, the duration set at 10 mins, and the accounting set as cumulative, these criteria are met once the patient's SpO₂ has fallen equal to or below 86% for 10 cumulative minutes. As another example, with respect to the high limit, with the high limit deviation threshold set at +4%, the duration set at 30 mins, and the accounting set as consecutive, these criteria are met once the patient's SpO₂ has risen equal to or above 92% and has been maintained at such a level for at least 30 mins.

The action settings 360 correspond to the recommended action provide via an alert or notification or the action to be taken in a feedback-based system once the above-defined criteria are met. In particular, the action settings 360 may provide for an increase or decrease in the FiO₂ setting by a particular amount, e.g., “increase FiO₂ 5%,” or may trigger issuance of an alert/notification or report indicating the same. The result setting 370 indicates how the action, as determined by action setting 360, is carried out. For example, where “Auto Update” is provided, upon meeting the requisite criteria, the feedback-based system is enabled and processor 216 signals ventilator system 112 to, for example, increase the FiO₂ level 5%. Where the “Alert” setting is used, an audible, text-based, visual, and/or other suitable alert/notification or report is provided upon meeting the appropriate criteria. The alert may indicate the recommended action, as set via the action setting 360, or may provide a general alert. Either or both of the “Alert” and “Auto Update” settings may be selected, as desired. Additionally or alternatively, adjustment limits, set by the user, may also be provided to inhibit or warn against FiO₂ setting adjustments outside maximum or minimum settings, where multiple adjustments within a pre-determined time period are effected, or where a particular adjustment exceeds a pre-defined adjustment ranges (in either absolute terms or as a percentage of the pre-adjustment setting). These adjustment limits, although not shown, may likewise be accessible or settable via setting screen 350.

Built within the above-described framework may be multiple levels of control based upon various pre-defined parameters or pre-defined conditions associated with the patient's SpO₂. For example, a first set of setting parameters may trigger alerts, notifications, and/or warnings upon adjustment or when adjustment is recommended, while a second set of setting parameters may effect automatic adjustment of the FiO₂ setting or inhibit adjustment of the FiO₂ setting, where appropriate and as provided via the corresponding settings. In such a configuration, several settings display pages may be accessible, allowing setting of the desired parameters for each level of control, e.g., a first settings page for the “alert” response and a second settings page for the “feedback” response.

Additional setting display pages may further be provided to allow different parameter ranges and/or conditions to be set for a particular range of FiO₂ settings. For example, where the FiO₂ setting is within a first range, adjusting the FiO₂ setting or alerting to the recommendation that the setting be adjusted may be provided in accordance with a first set of parameters or conditions, while adjusting and/or alerting regarding the FiO₂ setting may be provided in accordance with a second, different set of parameters or conditions where the FiO₂ setting is within a second, different range. As such, multi-level or dynamic parameter setting can be readily achieved. Further, pre-set or default settings may be automatically acquired for populating any or all of the above-noted setting fields based upon information available via system 10. For example, a different pre-set configuration may be provided for each of a plurality of situations corresponding to, for example, the reason(s) or condition(s) that necessitate a patient being on ventilation, biographical or physiological factors or information, user preference, facility protocol, etc. In particular, different configurations of settings may be desirable for ARDS patients, COPD patients, surgery patients, etc. As such, the various alerts, warnings, ranges, feedback controls, etc. may set in accordance with the particular needs of the patient.

The above-noted alerts/notifications may be provided in any suitable form, e.g., as a visual display, audible alert, data save, print-out, message (text, email, voicemail, etc.) and may be local and/or remote, e.g., transmitted across system 10, broadcast, etc. The alerts and/or notifications provided at the various stages may differ in type, volume, size, length, etc., to indicate a severity or urgency of that particular notification and well as relative to other notifications.

In addition to alerts and notifications, reports may be automatically generated by processor 216 and transmitted or printed in response to fulfillment of particular conditions, depending on protocol or user-settings. These automatically generated repots may include, for example, reproductions of graphs 312, 322, or may extract data from graphs 312, 322 to create a text-based report summarizing the data. Other information received, generated, and/or stored by system 10 may also be utilized in the reports. The reports can then be used to fulfill reporting requirements, for studies, or other internal (or external) purposes without the need for medical personnel to manually fill out the required paper work. For example, reports may be generated and transmitted to an appropriate database for inclusion in the patient's EMR file and/or an ADT electronic file. Reports may likewise be generated for collecting lab data for use in a particular laboratory study or studies. As an alternative to or in addition to automatically-generated reports, report requests may be generated in accordance with one or more of the alerts or notifications, thus providing medical personnel with the option of generating a report upon receipt of the particular alert or notification. Further still, manual report requests may be initiated for generating a report for a particular purpose and/or at a particular time, as needed, e.g., to review a user's decision to change the FiO₂ level based upon SpO₂ data, analyze trends to determine optimal parameter settings and/or protocols, etc.

Turning now to FIG. 5, in conjunction with FIGS. 1 and 2, a display screen 400 in accordance with embodiments of the present disclosure is show displaying exemplary information as output by UI 218. Display screen 400 may represent display 118 of ventilator system 112 (or other patient care device 110), display 132 of bedside device 130, or a monitor, display, etc. of any of remote devices 170. That is, depending on the configuration of system 10, the user may view display screen 400 on one of the patient care devices 110, patient monitoring devices 120, 121, bedside devices 130, remote devices 170, or any other suitable device. Display screen 400, as will be detailed below, incorporates the above-described features of monitoring, compiling, and outputting information indicating the relationship between the FiO₂ setting of the ventilator and the patient's SpO₂ into systems and methods for monitoring a patient on mechanical ventilation for risks of VAEs and for observing the occurrence of VAEs.

Display screen 400 includes first, second, third, fourth, and fifth graph image areas 402, 410, 420, 430, 440 for graphically displaying current and trending information relating to PEEP, FiO₂, temperature, WBC count, and SpO₂, respectively. The orientation, ordering, and/or relative positioning of graph image areas 402, 410, 420, 430, 440 may be selected and/or changed in accordance with user preference. Patient information, e.g., biographical information, drug administration schedule information and/or days-on-ventilator information, is also displayed on display screen 400 via text boxes 452, 454, 456. Display screen 400 further includes a status indication area 460 for displaying a status indicator 462 based upon current and/or trending information, e.g., PEEP, FiO₂, temperature, WBC count, SpO₂, and other patient information. Each of the above-noted features of display screen 400 will be described in detail, in turn, below.

First graph image area 402 is configured for displaying an SpO₂ graph 404 representing the patient's measured SpO₂ (as a percentage) as a function of time. SpO₂ graph 404 may include any of the features detailed above with respect to SPO₂ graph 312 (FIG. 3). SpO₂ measurements may be taken at pre-determined intervals or continuously, e.g., using pulse oximeter 126. In use, SpO₂ measurements received by pulse oximeter 126 are transmitted to processor 216 in real-time (or are transmitted to storage 212, for processing at a later time) for processing and output as a graphical representation for display in fifth graph image area 402 of display screen 400. An icon 490 positioned next to both FiO₂ graph 414 and SpO₂ graph 404, is provided for allowing a user to selectively view a larger and/or more detailed view of these two graphs 414, 404. For example, selecting icon 490 may switch from display screen 400 to display screen 300 (FIG. 3).

Second graph image area 410 is configured for displaying a FiO₂ graph 414 representing the FiO₂ level (as a percentage) set in ventilator system 112 as a function of time. FiO₂ graph 414 may include any of the features detailed above with respect to FiO₂ graph 322 (FIG. 3), and vice versa. FiO₂ graph 414 is shown including an actual FiO₂ indicator line 416 (the broken line) and a daily low FiO₂ indicator line 418 (the solid line) superimposed on one another and, similarly as above, may include shading or coloring in accordance with a coding protocol and/or alert indicators 419 at particular points of concern. FiO₂ level information set in ventilator system 112 is transmitted to processor 216 in real-time (or is transmitted to storage 212, for processing at a later time) for processing and output as a graphical representation for display in second graph image area 410 of display screen 400.

Third graph image area 420 is configured for displaying a PEEP graph 424 representing the PEEP level (measured in cmH₂O) set in ventilator system 112 as a function of time. PEEP graph 424 includes an actual PEEP indicator line 426 (the broken line) and a daily low PEEP indicator line 428 (the solid line) superimposed on one another. As shown, the area under the daily low PEEP indicator line 428 may also be shaded or colored in accordance with a coding protocol, and/or alert indicators 429 may be provided in graph 424 at particular points of concern. PEEP level information set in ventilator system 112 is transmitted to processor 216 in real-time (or is transmitted to storage 212, for processing at a later time) for processing and output as a graphical representation for display in third graph image area 420 of display screen 400.

Fourth graph image area 430 is configured for displaying a temperature graph 434 representing the patient's measured temperature (in ° C.) as a function of time. Temperature measurements may be taken at pre-determined intervals or continuously, e.g., using temperature sensor 122. As noted above, portions of temperature graph 434 may be shaded or colored in accordance with a coding protocol. Temperature measurements received by temperature sensor 122 are transmitted to processor 216 in real-time (or are transmitted to storage 212, for processing at a later time) for processing and output as a graphical representation for display in third graph image area 430 of display screen 400.

Fifth graph image area 440 is configured for displaying a graph 444 representing the patient's WBC count (in WBCs/mcL) as a function of time. WBC count measurements are taken at pre-determined intervals, e.g., once per day, using standard blood analysis techniques. The WBC count measurement is input into, for example, bedside device 130 or one of remote devices 170, for transmission processor 216 (or storage 212) for processing and output as a graphical representation for display in fourth graph image area 440 of display screen 400. Portions of WBC count graph 444 may be shaded or colored in accordance with a coding protocol.

Text boxes 452, 454, 456 display relevant patient information including biographical information, e.g., the patient's name, age, etc., and hospital record information, e.g., the patient's drug administration schedule, the number of days-on-ventilator, or other relevant information. The information displayed in text boxes 452, 454, 456 of display screen 400 may be input, for example, via bedside device 130, one of remote devices 170, or may be pulled from electronic files or records in other locations via server(s) 140, 150, 160.

Status indication area 460 of display screen 400 is provided for displaying a status indicator 462 as a function of the current and trending PEEP levels, FiO₂ levels, patient temperature, patient SpO₂, patient WBC count, and/or other information. Status indicator 462 may indicate a patient's risk level of experiencing a VAE, that a VAE has occurred in the past, and/or that a VAE is ongoing. More specifically, depending on the current and/or trending PEEP levels, FiO₂ levels, patient SpO₂, patient temperature, and/or patient WBC count, and in accordance with other information, a risk status or current status is determined and displayed in status indication area 460. For example, status indication area 460 may remain blank where the above-noted information indicates that the patient is in stable condition with a relatively low risk of VAE; a medium-risk indicator, e.g., a yellow icon, may be provided where current and trending information indicates that the patient's risk of experiencing a VAE is increasing; and a red icon may be provided where a VAE has occurred, is ongoing, or where the patient is at a high risk for experiencing a VAE. Other indicator systems are also contemplated.

Referring generally to FIGS. 1, 2, and 5, certain parameters, conditions, and/or sensed data, alone or in combination with other parameters, conditions, and/or sensed data, have been found to indicate an increased risk that a patient may suffer a VAE, or that a VAE is occurring, about to occur, or has occurred. In particular, it has been found that the need for sustained increase in the FiO₂ level set in ventilator system 112, e.g., to account for a patient's increasing oxygenation needs as indicated by SpO₂ levels, after a stable (or decreasing) period may be caused by a VAE. This situation will be detailed below.

The need for sustained increase in the PEEP level set in ventilator system 112, e.g., to account for a patient's increased oxygenation needs, after a stable (or decreasing) period may also be caused by a VAE. Further, the need to start a patient on a new antimicrobial agent, or drug, e.g., to combat a potential infection, may be caused by a VAE. An increase in patient body temperature and/or WBC count may also indicate a potential infection, illness, or other condition which may be caused by a VAE. Such situations are detailed in U.S. patent application Ser. No. 13/768,769, previously incorporated by reference herein.

With respect to increasing the FiO₂ level, as detailed above, this step is often taken in response to a drop (or too low) in SpO₂. That is, such a step is reactionary. Thus, rather than awaiting the FiO₂ change, which is often necessitated by SpO₂ data, the SpO₂ data can be viewed directly on display screen 400 for early ascertainment of whether a patient is trending towards a potential VAE, whether or not the FiO₂ level has been changed. Alternatively or additionally, in embodiments where the SpO₂ level and FiO₂ setting are part of a feedback-based control loop, and, thus, the FiO₂ setting is adjusted automatically in response to SpO₂ changes, the user can be sure the FiO₂ data is up-to-date and view display 400 to readily ascertain whether a sustained increase has occurred or is in the midst of occurring.

With respect to utilizing SpO₂ data in order to ascertain whether a patient is trending towards a potential VAE, is in the midst of a VAE, or has suffered a VAE, an independent set of parameter settings may be utilized. That is, one or more sets of parameter settings may be utilized, as detailed above, for automatically adjusting the FiO₂ setting or for producing alerts/notification indicating that such adjustment may be warranted, an independent parameter setting may be utilized for alerting a user regarding a potential, ongoing, or occurred VAE and/or for reporting such VAEs. For example, depending on a particular situation, facility protocol, etc., the parameter settings for adjusting the FiO₂ setting (automatically or via alerting a user to manually adjust the setting) may be different from the parameter settings required for collecting data and reporting the occurrence of a VAE to the CDC or other reporting agency, for studies, or other internal (or external) purposes. Thus, although provided within a single system, these two features, namely automatic or alert-recommended adjustment of FiO₂ and VAE reporting/alerting features, need not utilize the same parameter settings and can remain independent of one another.

While several embodiments of the disclosure have been shown in the drawings and described in detail hereinabove, it is not intended that the disclosure be limited thereto, as it is intended that the disclosure be as broad in scope as the art will allow. Therefore, the above description and appended drawings should not be construed as limiting, but merely as exemplifications of particular embodiments. Those skilled in the art will envision other modifications within the scope and spirit of the claims appended hereto.

What is claimed is:

1. A system for monitoring a patient on ventilation, comprising:
a ventilator system configured to provide mechanical ventilation in accordance with an FiO₂ setting;
a pulse oximeter configured to measure a patient's SpO₂;
an information processing system communicatively coupled to the ventilator system and the pulse oximeter, the information processing system configured to receive, process, and output FiO₂ setting information and patient SpO₂ data from the ventilator system and the pulse oximeter, respectively; and
a display device configured to display the FiO₂ setting information and patient SpO₂ data graphically as a function of time.

2. The system according to claim 1, wherein the information processing system is further configured to output an alert or notification in response to a change in the patient's SpO₂.

3. The system according to claim 2, wherein the alert or notification includes a recommended adjustment of the FiO₂ setting.

4. The system according to claim 1, wherein the information processing system is further configured to control adjustment of the FiO₂ setting in response to a change in the patients SpO₂.

5. The system according to claim 1, wherein the information processing system is further configured to at least one of receive and store a baseline SpO₂, high limit SpO₂ threshold settings, and low limit SpO₂ threshold settings.

6. The system according to claim 5, wherein the information processing system is further configured to output an alert or notification where the patient's SpO₂ reaches or exceeds the high or low limit SpO₂ threshold settings.

7. The system according to claim 5, wherein the information processing system is further configured to control adjustment of the FiO₂ setting where the patient's SpO₂ reaches or exceeds the high or low limit SpO₂ threshold settings.

8. The system according to claim 1, wherein the display device is configured to provide color-coding associated with the graphical display of the FiO₂ setting information.

9. The system according to claim 8, wherein the color-coding is associated with the patient SpO₂ data.

10. The system according to claim 1, wherein the graphical display of the patient SpO₂ data includes indicators indicating where adjustments to the FiO₂ setting have been made.

11. The system according to claim 1, wherein the information processing system is further configured to utilize the patient SpO₂ data to determine a potential VAE.

12. A method of monitoring a patient on a ventilator, comprising the steps of:
obtaining data indicative of an FiO₂ setting of a ventilator;
obtaining data indicative of a patient's SpO₂; and
outputting information indicating a relationship between the data indicative of the FiO₂ setting of the ventilator and the data indicative of the patient's SpO₂.

13. The method according to claim 12, wherein the step of outputting the information includes displaying both the data indicative of the FiO₂ setting of the ventilator and the data indicative of the patient's SpO₂ graphically as a function of time.

14. The method according to claim 12, wherein the step of outputting information includes providing an alert or a notification recommending an adjustment of the FiO₂ setting based upon the data indicative of the patient's SpO₂.

15. The method according to claim 14, wherein the alert or notification is further based upon a baseline SpO₂ and SpO₂ threshold settings.

16. The method according to claim 12, wherein the step of outputting information includes outputting a control signal for adjusting the FiO₂ setting based upon the data indicative of the patient's SpO₂.

17. The method according to claim 16, wherein outputting the control signal is further based upon a baseline SpO₂ and SpO₂ threshold settings.

18. A non-transitory computer-readable storage medium encoded with a program that, when executed by a processor, causes the processor to perform the steps of:
obtaining data indicative of an FiO₂ setting of a ventilator;
obtaining data indicative of a patient's SpO₂; and
outputting at least one of an alert or notification and a control signal for adjusting the FiO₂ setting based upon both the data indicative of the FiO₂ setting of the ventilator and the data indicative of the patient's SpO₂.

19. The medium according to claim 18, wherein the step of outputting is further based upon a baseline SpO₂ and SpO₂ threshold settings.

20. The medium according to claim 18, wherein the processor is further caused to perform the step of displaying both the data indicative of the FiO₂ setting of the ventilator and the data indicative of the patient's SpO₂ graphically as a function of time.
